(19) United States
(12) Patent Application Publication
(10) Pub. No.: US 2007/0129741 A1
(43) Pub. Date: Jun. 7, 2007

(54) ARTIFICIAL ANTERIOR CHAMBER FOR USE IN KERATOPLASTY

(76) Inventor: Philip Douglas WESTON, North Yorkshire (GB)

Correspondence Address: GARVEY SMITH NEHRBASS & NORTH, LLC, LAKEWAY 3, SUITE 3290, 3838 NORTH CAUSEWAY BLVD., METAIRIE, LA, 70002, US

(21) Appl. No.: 11/564,440

(22) Filed: Nov. 29, 2006

(30) Foreign Application Priority Data
Dec. 7, 2005 (GB) 0524965.1

(51) Int. Cl. A61F 9/00

(52) U.S. Cl. 606/166

(57) ABSTRACT

There is disclosed a device for retaining a donor cornea when cutting a donor button. The device comprises a base unit having a hollow cylindrical pedestal projecting therefrom, the pedestal having a central axis and an end remote from the base unit defining an annular basal surface adapted to receive a donor cornea. There may be provided cross-hair means for visually indicating a position of the central axis of the pedestal, and removable retaining means adapted to fit snugly over the pedestal so as to retain the donor cornea securely in position on the end of the pedestal.

### CROSS-REFERENCE TO RELATED APPLICATIONS

Priority of U.K. Patent Application No. 0524965.1, filed 7 Dec. 2005, incorporated herein by reference, is hereby claimed.

### STATEMENT REGARDING FEDERALLY SPONSORED RESEARCH OR DEVELOPMENT

Not applicable.

### REFERENCE TO A “MICROFICHE APPENDIX”

Not applicable.

### BACKGROUND OF THE INVENTION

1. Field of the Invention

The present invention relates to an anterior chamber for use in keratoplasty, and in particular to a device adapted for facilitating the cutting of a donor button from the epithelial surface of a donor cornea harvested from a cadaver.

2. General Background of the Invention

In the field of eye surgery, it is sometimes necessary to perform a corneal transplant by replacing a diseased or damaged cornea of a patient with a donor cornea harvested from an immunologically compatible cadaver. This is generally achieved by removing at least a central portion of the diseased or damaged cornea from a patient's eye using a trephine, using a corneal punch to cut an equivalent portion from a donor cornea (this portion being known in the art as a donor button), and then inserting the donor button into the space on the surface of the patient's eye left vacant by removal of the diseased or damaged portion of the patient's original cornea.

The procedure is generally known as keratoplasty. In fill penetrating keratoplasty, the entire depth of a patient's cornea is removed and replaced with a donor button cut to the full depth of the donor cornea. In partial or deep lamellar keratoplasty, by contrast, only an upper layer of the patient's cornea is removed, and replaced with a corresponding layer of a donor cornea. In endothelial lamellar keratoplasty, a layer of the endothelium is replaced, while in anterior lamellar keratoplasty, a layer of the stroma is replaced.

Various corneal punches are known in the art for cutting corneal donor buttons for use in surgical keratoplasty techniques. One example is that of EP1273278, which comprises a base having a well in which a donor cornea is placed, and a guide in which a trephine may be inserted so as to cut the donor button. In prior art punches, however, it can be difficult to ensure that the donor cornea is properly centred before cutting the donor button. Moreover, when it is desired to keep the donor cornea flooded with a balanced salt solution during cutting of the button, there is often significant leakage of the salt solution.

### BRIEF SUMMARY OF THE INVENTION

According to a first aspect of the present invention, there is provided a device for retaining a donor cornea when cutting a donor button therefrom, the device comprising: a base unit having a hollow cylindrical pedestal projecting therefrom, the pedestal having a central axis and an end remote from the base unit defining an annular basal surface adapted to receive a donor cornea; means for visually indicating a position of the central axis of the pedestal; and removable retaining means adapted to fit snugly over the pedestal so as to retain the donor cornea securely in position on the end of the pedestal.

The removable retaining means may comprise a collar having a cylindrical portion adapted to fit snugly over the pedestal, with an inwardly-facing flange at one end of the cylindrical portion adapted to cooperate with the annular basal surface of the pedestal so as to clamp the donor cornea thereto. An opposed end of the cylindrical portion may have an outwardly-facing flange so as to allow the retaining means to be clamped to the base unit, for example by way of a threaded compression ring adapted to screw down onto a threaded portion of the base unit provided about the pedestal at an end thereof proximate to the base unit.

The threaded portion of the base unit may include an alignment pin or hole adapted to be received by or to receive, respectively, a corresponding alignment hole or peg provided on the removable retaining means.

The means for visually indicating the position of the central axis of the pedestal may comprise at least a pair of cross-hairs, and possibly three or more two cross-hairs, located in the pedestal, preferably near the annular basal surface thereof.

The provision of the cross-hairs allows a surgeon quickly and easily to ensure that a donor cornea is properly centred on the basal surface of the pedestal before (and during) positioning of the removable retaining means.

Once the donor cornea is securely retained by the device of the present invention, a standard trephine may be applied (through the cylindrical portion of the collar of the removable retaining means) so as to cut a donor button. Alternatively, laser cutting techniques may be applied to cut the donor button.

In particularly preferred embodiments, the base unit is hollow and is provided with an inlet for attachment to a liquid supply, for example a supply of balanced salt solution. The inlet may include a valve and be adapted for connection to a pipeline. Such inlets are often known as luer connectors. In use, the base unit, and hence the pedestal, can be filled with liquid so as to apply a precisely controlled fluid pressure to the underside of the donor corneal graft and thus give it a predetermined degree of curvature prior to cutting the button therefrom. Moreover, the liquid helps to keep the donor graft hydrated.

In these preferred embodiments, an elastomeric O-ring is preferably provided at the annular basal surface of the pedestal so as to ensure a leak-free compressive connection between the removable retaining means and the pedestal. Alternatively, an O-ring may be provided at the inwardly-facing flange of the removable retaining means to perform the same function. The O-ring may be made of silicone or any other surgically-compatible elastomeric material.

According to a second aspect of the present invention, there is provided a device for retaining a donor cornea when cutting a donor button therefrom, the device comprising: a hollow base unit having a hollow cylindrical pedestal projecting therefrom, the pedestal having a central axis and an end remote from the base unit defining an annular basal surface adapted to receive a donor cornea; removable retaining means adapted to fit snugly over the pedestal so as to retain the donor cornea securely in position on the end of the pedestal; the base unit having an inlet for connection to a liquid supply; and elastomeric sealing means adapted for ensuring a leak-free connection between the removable retaining means and the annular basal surface of the pedestal when a donor cornea is in place and the retaining means is fully fitted over the pedestal.

Advantageously, there is further provided means for visually indicating a position of the central axis of the pedestal as described in relation to the first aspect of the present invention.

All components may be made of surgical grade plastics materials and/or from surgical grade metal materials. In a currently preferred embodiment, the base unit and pedestal are integrally moulded from a plastics material, the compression ring is also moulded from a plastics material, and the retaining means is made of stainless steel.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 shows an embodiment of the present invention in disassembled condition;

FIG. 2 shows the embodiment of FIG. 1 in a partially assembled condition; and

FIG. 3 shows the embodiment of FIG. 1 in a fully assembled condition.

### DETAILED DESCRIPTION OF THE INVENTION

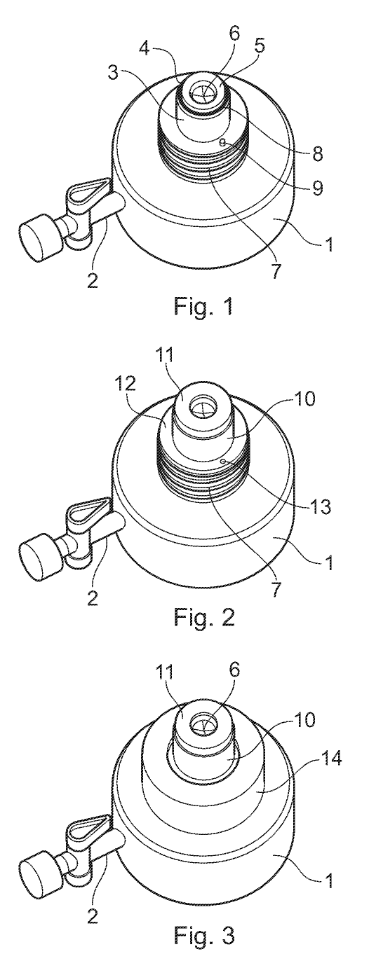

FIG. 1 shows a device of an embodiment of the present invention comprising a hollow, plastics moulded base unit 1 with a luer connection 2 for connection to a supply of balanced salt solution. A hollow, cylindrical pedestal 3 is integrally moulded with the base unit 1 and projects therefrom. The pedestal 3 has an end 4 remote from the base unit 1, which end 4 defines an annular basal surface 5 adapted to receive a donor cornea (not shown). The pedestal 3 is provided with cross-hairs 6 near its end 4, the cross-hairs 6 indicating the location of a central axis of the pedestal 3. The annular basal surface is provided with a silicone O-ring seal 8 about its periphery. A screw thread 7 is provided at a base of the pedestal 3, and an alignment peg 9 is also provided at the base.

FIG. 2 shows a removable, cylindrical retaining collar 10 mounted on the pedestal 3. The collar 10 has an inwardly-facing flange 11 at its upper end, and an outwardly-facing flange 12 at its lower end. The flange 11 cooperates with the basal surface 5 of the pedestal 3 so as securely to clamp a donor cornea therebetween. The O-ring seal 8 (not visible in FIG. 2) helps to ensure a leak-proof seal between the collar 10 and the pedestal 3. The flange 12 includes an alignment hole 13 that receives the alignment peg 9 of the base of the pedestal 3.

As shown in FIG. 3, the collar 10 is secured to the pedestal 3 of the base unit 1 by way of a compression ring 14 that screws down onto the screw thread 7 of the base of the pedestal 3.

In use, the luer connector 2 is connected to a supply of balanced salt solution (BSS). A corneal graft (not shown) is harvested from a cadaver by known techniques, and its endothelial surface is coated with a viscoelastic coating. The hollow base unit 1 is filled with BSS by opening the luer connector 2. The donor graft is then centred on the basal surface 5 of the pedestal 3 using the cross-hairs 6 as a guide. The collar 10 is then carefully located over the pedestal 3, and lowered thereupon such that the alignment peg 9 and hole 13 engage with each other. The collar 10 is pressed down firmly so as to ensure a tight seal between the flange 11, the basal surface 5, the O-ring seal 8 and the donor graft, and the compression ring 14 is then fitted and screwed down tightly.

A desired BSS fluid pressure is then applied by careful operation of the luer connector 2, and/or by injecting air or other fluid into the base unit 1 by way of a syringe, and/or by compressing the base unit 1 using an infusion pump, and/or by raising or lowering the device relative to the fluid source.

When the desired pressure is obtained, any suitable cutting technique (e.g. application of a trephine or a laser) is used to cut the donor button.

Advantageously, the entire unit is configured as a disposable, single-use article supplied in sterile packaging.

Parts List:

The following is a list of parts and materials suitable for use in the present invention:
1 base unit
2 luer connection
3 pedestal
4 end
5 basal surface
6 cross-hairs
7 screw thread
8 seal
9 alignment peg
10 collar
11 flange
12 flange
13 hole
14 compression ring

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims:

1. A device for retaining a donor cornea when cutting a donor button therefrom, the device comprising:
a base unit having a hollow cylindrical pedestal projecting therefrom, the pedestal having a central axis and an end remote from the base unit defining an annular basal surface adapted to receive a donor cornea;
means for visually indicating a position of the central axis of the pedestal; and
removable retaining means adapted to fit snugly over the pedestal so as to retain the donor cornea securely in position on the end of the pedestal.

2. A device as claimed in claim 1, wherein the removable retaining means comprises a collar having a cylindrical portion adapted to fit snugly over the pedestal, and an inwardly-facing flange at one end of the cylindrical portion adapted to cooperate with the annular basal surface of the pedestal.

3. A device as claimed in claim 2, wherein an opposed end of the cylindrical portion has an outwardly-facing flange.

4. A device as claimed in claim 1, further comprising means for locking the retaining means to the base unit.

5. A device as claimed in claim 1, further comprising means for maintaining the removable retaining means in rotational registration with the base unit when mounted thereon.

6. A device as claimed in claim 1, wherein the means for visually indicating the position of the central axis of the pedestal comprises at least a pair of cross-hairs.

7. A device as claimed in claim 6, wherein the cross-hairs are located in the pedestal, preferably near the annular basal surface thereof.

8. A device as claimed in claim 1, wherein the base unit is hollow and is provided with an inlet for attachment to a liquid supply.

9. A device as claimed in claim 8, wherein an elastomeric O-ring is provided at the annular basal surface of the pedestal.

10. A device as claimed in claim 8, wherein an elastomeric O-ring is provided in the removable retaining means.

11. A device for retaining a donor cornea when cutting a donor button therefrom, the device comprising:
a hollow base unit having a hollow cylindrical pedestal projecting therefrom, the pedestal having a central axis and an end remote from the base unit defining an annular basal surface adapted to receive a donor cornea;
removable retaining means adapted to fit snugly over the pedestal so as to retain the donor cornea securely in position on the end of the pedestal;
the base unit having an inlet for connection to a liquid supply; and
elastomeric sealing means adapted for ensuring a leak-free connection between the removable retaining means and the annular basal surface of the pedestal when a donor cornea is in place and the retaining means is fully fitted over the pedestal.

12. A device as claimed in claim 11, further comprising means for visually indicating a position of the central axis of the pedestal.

13. A device as claimed in claim 12, wherein the means for visually indicating the position of the central axis of the pedestal comprises at least a pair of cross-hairs.

14. A device for retaining a donor cornea when cutting a donor button therefrom, substantially as hereinbefore described with reference to or as shown in the accompanying drawings.
